(19) United States
(12) Patent Application Publication
(10) Pub. No.: US 2025/0205038 A1
(43) Pub. Date: Jun. 26, 2025

(54) TISSUE SCAFFOLD AND METHOD OF MANUFACTURING THE SAME

(71) Applicant: Industrial Technology Research Institute, Hsinchu (TW)

(72) Inventors: Chih-Chieh Huang, Zhunan Township (TW); Lih-Tao HSU, Lujhu Township (TW); Cheng-Yi WU, Zhubei City (TW); Meng-Hsueh LIN, Taoyuan City (TW); Hui-Ting HUANG, Toufen City (TW); Chen-Hsuan LIN, Zhudong Township (TW); Yi-Hung WEN, Hsinchu City (TW); Fang-Chieh CHANG, Zhudong Township (TW); Hsin-Hsin SHEN, Zhudong Township (TW); Pei-I TSAI, Hsinchu City (TW); Jun-Jae HUANG, Toufen City (TW)

(73) Assignee: Industrial Technology Research Institute, Hsinchu (TW)

(21) Appl. No.: 18/397,703

(22) Filed: Dec. 27, 2023

(30) Foreign Application Priority Data
Dec. 20, 2023 (TW) 112149722

(51) Int. Cl. A61F 2/08; A61F 2/00

(52) U.S. Cl. CPC A61F 2/08; A61F 2002/0068; A61F 2002/0081; A61F 2210/0076; A61F 2240/001; A61F 2250/0023

(57) ABSTRACT

A tissue scaffold is provided. The tissue scaffold includes a textile formed by interweaving a plurality of warp yarns and a plurality of weft yarns. The textile includes a first region and a second region, and the second region is adjacent to the first region. The plurality of warp yarns have different diameters in the first region and the second region. The textile has a plurality of pores, and the size of each of the plurality of pores is between 100 μm and 800 μm. A method of manufacturing the aforementioned tissue scaffold is also provided.

### CROSS REFERENCE TO RELATED APPLICATIONS

This application claims priority of Taiwan Patent Application No. 112149722, filed on Dec. 20, 2023, the entirety of which is incorporated by reference herein.

### TECHNICAL FIELD

The present disclosure is related to a biomedical material used in tissue engineering, and in particular it is related to a high-strength tissue scaffold and a method of manufacturing the same.

### BACKGROUND

Tear or rupture injuries of tendons or ligaments are common clinical sports injuries. In particular, when such an injury occurs in the cruciate ligament in the knee joint, the ligament tissue has no ability to heal naturally after rupture due to its limited biological environment and blood supply, and requires surgical reconstruction. For example, artificial ligaments can be transplanted into the affected area for tendon or ligament reconstruction.

However, current artificial ligament products often cause adverse reactions such as synovitis, arthritis, or rupture after long-term implantation. Histological analysis of the reasons for the failure of artificial ligament reconstruction shows that most commercially available artificial ligament products focus on providing mechanical strength repairs and lack the ability to integrate with surrounding tissues. Only a small number of fibroblasts and blood vessels grow in the affected area. Under long-term use, implants gradually wear out, lose structural integrity, and eventually loosen or break.

In view of the foregoing, further development of high-strength tissue scaffolds with both mechanical strength and tissue compatibility to provide a place for regenerative tissue cell attachment and stable proliferation is still one of the research topics in related fields.

### SUMMARY

In accordance with some embodiments of the present disclosure, a tissue scaffold is provided. The tissue scaffold includes a textile formed by interweaving a plurality of warp yarns and a plurality of weft yarns. The textile includes a first region and a second region, and the second region is adjacent to the first region. The plurality of warp yarns have different diameters in the first region and the second region. The textile has a plurality of pores, and the size of each of the plurality of pores is between 100 micrometers and 800 micrometers.

In accordance with further embodiments of the present disclosure, a method of manufacturing a tissue scaffold is provided. The method includes forming a textile. Forming the textile includes the following steps: providing a plurality of warp yarns and performing warping, and the plurality of warp yarns have at least two different diameters; providing a plurality of weft yarns and performing beat-up; and interweaving the plurality of warp yarns and the plurality of weft yarns to form the textile. Furthermore, the textile includes a first region and a second region adjacent to the first region, and the plurality of warp yarns have different diameters in the first region and the second region. The textile has a plurality of pores, and the size of each of the plurality of pores is between 100 micrometers and 800 micrometers.

A detailed description is given in the following embodiments with reference to the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present invention can be more fully understood by reading the subsequent detailed description and examples with references made to the accompanying drawings, wherein:

FIG. 1 is a schematic diagram of a tissue scaffold in accordance with an embodiment of the present disclosure;

FIG. 2 is a schematic diagram of a tissue scaffold in accordance with an embodiment of the present disclosure;

FIG. 3A is a schematic diagram of a tissue scaffold with a rolled-strip shape in accordance with an embodiment of the present disclosure;

FIG. 3B is a schematic diagram of a tissue scaffold with a sheet/folded shape in accordance with an embodiment of the present disclosure;

FIG. 4A is the cell proliferation analysis result of the first region of a tissue scaffold prepared in accordance with the embodiments of the present disclosure;

FIG. 4B is the cell morphology analysis result of the first region of a tissue scaffold prepared in accordance with the embodiments of the present disclosure;

FIG. 5A is the cell proliferation analysis result of the second region of a tissue scaffold prepared in accordance with the embodiments of the present disclosure;

FIG. 5B is the cell morphology analysis result of the second region of a tissue scaffold prepared in accordance with the embodiments of the present disclosure;

FIG. 6 is the analysis result of cell attachment rates of tissue scaffolds with different pore sizes in accordance with the embodiments of the present disclosure;

FIG. 7 is the analysis result of cell proliferation rates of tissue scaffolds with different pore sizes in accordance with the embodiments of the present disclosure;

FIG. 8A is the cell proliferation analysis result of a tissue scaffold with a multi-layer structure in accordance with the embodiments of the present disclosure;

FIG. 8B is the cell morphology analysis result of a tissue scaffold with a multi-layer structure in accordance with the embodiments of the present disclosure;

FIG. 9 is the cell proliferation analysis result of a tissue scaffold in accordance with a Comparative Example;

FIG. 10A to FIG. 10C are the tensile testing results of tissue scaffolds prepared in accordance with the embodiments of the present disclosure;

FIG. 11A and FIG. 11B are the X-ray analysis results of tissue scaffolds prepared in accordance with the embodiments of the present disclosure as an artificial ligament implanted in an animal body for 1 month;

FIG. 12A is the visual observation result the tissue scaffold prepared in the embodiment of the present disclosure as an artificial ligament in an animal body after 3 months of implanting;

FIG. 12B is the analysis result of electron microscope of a tissue scaffold prepared in accordance with the embodiments of the present disclosure as an artificial ligament implanted in an animal for 3 months.

### DETAILED DESCRIPTION

The high-strength tissue scaffold and its manufacturing method of the present disclosure are described in detail in the following description. It should be understood that in the following detailed description, for purposes of explanation, numerous specific details and embodiments are set forth in order to provide a thorough understanding of the present disclosure. The specific elements and configurations described in the following detailed description are set forth in order to clearly describe the present disclosure. It will be apparent that the exemplary embodiments set forth herein are used merely for the purpose of illustration and not the limitations of the present disclosure.

In the following description, the terms “about” and “substantially” typically mean+/−10% of the stated value, or typically +/−5% of the stated value, or typically +/−3% of the stated value, or typically +/−2% of the stated value, or typically +/−1% of the stated value or typically +/−0.5% of the stated value. The stated value of the present disclosure is an approximate value. When there is no specific description, the stated value includes the meaning of “about” and “substantially”. In addition, the term “in a range from the first value to the second value” or “in a range between the first value and the second value” means that the range includes the first value, the second value, and other values in between.

Unless defined otherwise, all technical and scientific terms used herein have the same meaning as commonly understood by one of ordinary skill in the art to which this disclosure belongs. It should be appreciated that, in each case, the term, which is defined in a commonly used dictionary, should be interpreted as having a meaning that conforms to the relative skills of the present disclosure and the background or the context of the present disclosure, and should not be interpreted in an idealized or overly formal manner unless so defined.

In accordance with some embodiments of the present disclosure, a high-strength tissue scaffold is provide. The tissue scaffold includes a textile with a specific configuration, so that the tissue scaffold has both mechanical strength and tissue compatibility, can effectively load a weight and provide structural stability, and has good tissue compatibility allowing regenerated cells to attach to tissue scaffold and proliferate stably.

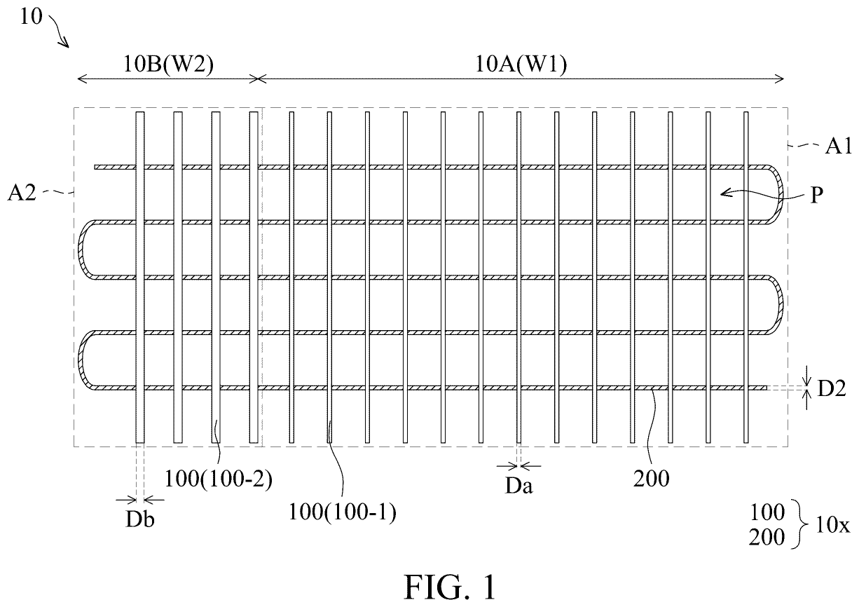

Please refer to FIG. 1, which is a schematic diagram of a tissue scaffold 10 in accordance with an embodiment of the present disclosure. As shown in FIG. 1, the tissue scaffold 10 includes a textile 10x formed by interweaving a plurality of warp yarns 100 and a plurality of weft yarns 200. In accordance with some embodiments, the warp yarns 100 are arranged substantially perpendicular to the weft yarns 200. Furthermore, the textile 10x includes a first region 10A and a second region 10B, and the second region 10B is adjacent to the first region 10A.

As used herein, the term “warp yarn” refers to the yarn that is extended along the longitudinal direction of the loom during weaving and serves as the main supporting structure in the textile, and is in the same direction as the tension when performing the telescopic action of the ligament. On the other hand, the term “weft yarn” refers to the yarn that is interlaced with or perpendicular to the warp yarn. In addition, as used herein, the term “textile” is formed by interweaving warp yarns and weft yarns in an interlaced or perpendicular manner, and the interlacing points thus formed can be continuously or discontinuously arranged, and the positions of interlacing points can be periodically selected or irregularly changed.

The warp yarns 100 have different diameters in the first region 10A and the second region 10B. For convenience of explanation, the warp yarn 100 located in the first region 10A is labeled 100-1, and the warp yarn 100 located in the second region 10B is labeled 100-2. In accordance with some embodiments, the warp yarn 100-1 has a first diameter Da in the first region 10A, the warp yarn 100-2 has a second diameter Db in the second region 10B, and the first diameter Da is smaller than the second diameter Db.

Specifically, in accordance with some embodiments, the first diameter Da may be between 50 denier and 200 denier, for example, 60 denier, 70 denier, 80 denier, 90 denier, 100 denier, 110 denier, 120 denier, 130 denier, 140 denier, 150 denier, 160 denier, 170 denier, 180 denier or 190 denier, but it is not limited thereto. In accordance with some embodiments, the second diameter Db may be between 500 denier and 2000 denier, for example, 600 denier, 700 denier, 800 denier, 900 denier, 1000 denier, 1100 denier, 1200 denier, 1300 denier, 1400 denier, 1500 denier, 1600 denier, 1700 denier, 1800 denier or 1900 denier, but it is not limited thereto.

Furthermore, in accordance with some embodiments, the filament number of the warp yarn 100-1 in the first region 10A may be between 1F and 20F, for example, 2F, 3F, 4F, 5F, 6F, 7F, 8F, 9F, 10F, 11F, 12F, 13F, 14F, 15F, 16F, 17F, 18F or 19F, but it is not limited thereto. In accordance with some embodiments, the filament number of warp yarn 100-2 in the second region 10B may be between 50F and 150F, or between 60F and 140F, or between 70F and 130F, for example, 80F, 85F, 90F., 95F, 100F, 105F, 110F, 115F, 120F or 125F, but it is not limited thereto.

In comparison, the weft yarns 200 have substantially the same diameter in the first region 10A and the second region 10B. In accordance with some embodiments, the diameter D2 of the weft yarn 200 may be between 50 denier and 200 denier, for example, 60 denier, 70 denier, 80 denier, 90 denier, 100 denier, 110 denier, 120 denier, 130 denier, 140 denier, 150 denier, 160 denier, 170 denier, 180 denier or 190 denier, but it is not limited thereto.

In accordance with some embodiments, the filament number of the weft yarn 200 may be between 1F and 20F, for example, 2F, 3F, 4F, 5F, 6F, 7F, 8F, 9F, 10F, 11F, 12F, 13F, 14F, 15F, 16F, 17F, 18F or 19F, but it is not limited thereto.

Furthermore, in accordance with some embodiments, the materials of the warp yarn 100 and the weft yarn 200 may include polyester, collagen, cellulose or a combination thereof, but they are not limited thereto. In accordance with some embodiments, the polyester may include polyethylene terephthalate (PET), polylactic acid (PLA), polycaprolactone (PCL), another suitable polyester, or a combination thereof, but it is not limited thereto. In addition, in accordance with some embodiments, weft yarn 200 may further include a bioceramic material. In accordance with some embodiments, the bioceramic material may include calcium phosphate, calcium sulfate, bioglass, or a combination thereof, but it is not limited thereto. In accordance with some embodiments, the calcium phosphate may include hydroxyapatite (HAp), tricalcium phosphate, or a combination thereof, but it is not limited thereto.

As described above, the textile 10x includes the first region 10A and the second region 10B adjacent to the first region 10A. In accordance with some embodiments, the first region 10A and the second region 10B occupy different widths or areas in the textile 10x. For example, the area A1 of the first region 10A is larger than the area A2 of the second region 10B. In accordance with some embodiments, the area ratio A1:A2 of the first region 10A and the second region 10B is between 1:1 and 20:1, for example, 2:1, 3:1, 4:1, 5:1, 6:1, 7:1, 8:1, 9:1, 10:1, 11:1, 12:1, 13:1, 14:1, 15:1, 16:1, 17:1, 18:1 or 19:1, but it is not limited thereto. In accordance with some embodiments, the width W1 of the first region 10A is greater than the width W2 of the second region 10B. In accordance with some embodiments, the width ratio W1:W2 of the first region 10A and the second region 10B is between 1:1 and 20:1, for example, 2:1, 3:1, 4:1, 5:1, 6:1, 7:1, 8:1, 9:1, 10:1, 11:1, 12:1, 13:1, 14:1, 15:1, 16:1, 17:1, 18:1 or 19:1, but it is not limited thereto.

It should be noted that the first region 10A formed by the interweaving of warp yarns 100 with relatively small diameters and weft yarns 200 can provide a good environment for cell attachment and growth; and the second region 10B formed by interweaving of warp yarns 100 with relatively large diameters and weft yarns 200 has good mechanical strength and can provide structural stability. When the textile 10x has the first region 10A and the second region 10B configured as described above, the tissue scaffold 10 can have both mechanical strength and tissue compatibility, and is an ideal material for artificial ligaments.

In addition, as shown in FIG. 1, the textile 10x has a plurality of pores P, and the size of the pores P may be between 100 micrometers and 800 micrometers (μm), for example, 200 micrometers, 300 micrometers, 400 micrometers, 500 micrometers, 600 micrometers or 700 micrometers, but it is not limited thereto. In accordance with some embodiments of the present disclosure, the size of the pore P may refer to the length or width of the pore.

Further, in accordance with some embodiments, the overall porosity of the textile 10x may be between 5% and 62%, for example, 10%, 15%, 20%, 25%, 30%, 35%, 40%, 45%, 50%, 55% or 60%, but it is not limited thereto. In accordance with some embodiments, the porosity of the textile 10x in the first region 10A is greater than or equal to the porosity of the textile 10x in the second region 10B. In accordance with some embodiments, the porosity of the textile 10x in the first region 10A may be between 5% and 65%, for example, 10%, 15%, 20%, 25%, 30%, 35%, 40%, 45%, 50%, 55% or 60%, but it is not limited thereto. In accordance with some embodiments, the porosity of the textile 10x in the second region 10B may be between 2% and 30%, for example, 5%, 10%, 15%, 20% or 25%, but is not limited thereto. In accordance with some embodiments of the present disclosure, the porosity of the textile refers to the ratio of the pore area to the textile area. Specifically, the overall porosity of the textile 10x is the ratio of the total area of the pores P to the total area of the textile 10x (e.g., the area A1 of the first region 10A plus the area A2 of the second region 10B); the porosity of the textile 10x in the first region 10A is the ratio of the area of the pores P in the first region 10A to the area of the first region 10A; and the porosity of the textile 10x in the second region 10B is the ratio of the area of the pores P in the second region 10B to the area of the second region 10B.

It should be noted that since the first region 10A has a relatively large porosity, it can provide a good environment for cell attachment and growth, while the warp yarns 100-2 of the second region 10B have good mechanical strength and can provide structural stability. When the textile 10x has the first region 10A and the second region 10B configured as described above, the tissue scaffold 10 can have both mechanical strength and tissue compatibility, and is an ideal material for artificial ligaments, which can improve its ability to integrate with surrounding tissue and help fibroblasts and blood vessels grow around the tissue scaffold.

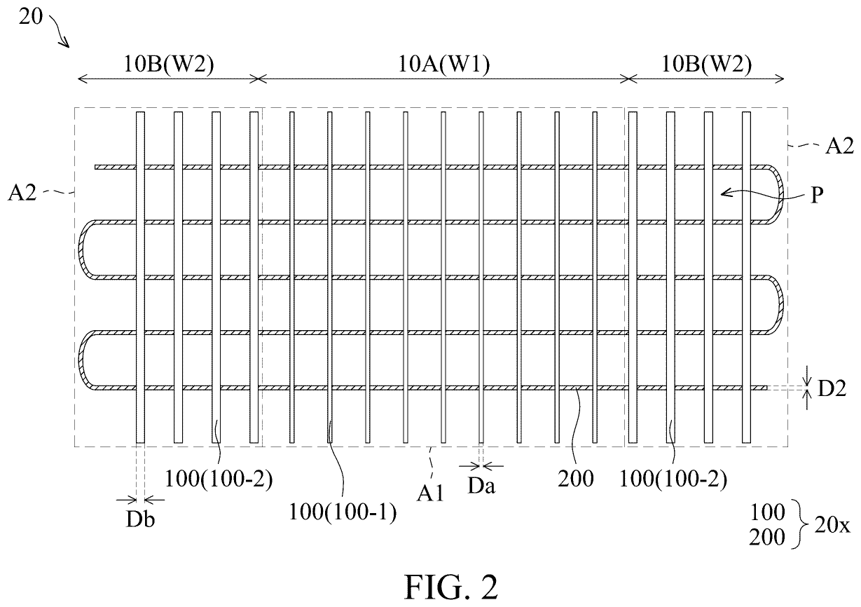

Next, please refer to FIG. 2, which is a schematic diagram of a tissue scaffold 20 in accordance with another embodiment of the present disclosure. It should be understood that the same or similar components or elements in the following description will be denoted by the same or similar numbers, and their materials and functions are the same or similar as those mentioned in the foregoing description, so they will not be repeated in the following description.

As shown in FIG. 2, the tissue scaffold 20 may further include another second region 10B, and the first region 10A may be located between the two second regions 10B. In accordance with some embodiments, the first regions 10A and the second regions 10B may be arranged in an alternating manner, and the numbers of the first regions 10A and the second regions 10B are not limited to those shown in the figures. According to different embodiments, the number of the first region 10A and the second region 10B can be adjusted according to needs.

Furthermore, it should be understood that although the tissue scaffold 20 includes another second region 10B, which is configured differently from the tissue scaffold 10, in the tissue scaffold 20, the width or area occupied by the first region 10A and any second region 10B in the textile 20x is also different. The first region 10A and any second region 10B also have a specific relationship as described in the aforementioned embodiments of the tissue scaffold 10. Specifically, the area A1 of the first region 10A may be larger than the area A2 of the second region 10B. In accordance with some embodiments, the area ratio A1:A2 of the first region 10A and the second region 10B is between 1:1 and 20:1. In accordance with some embodiments, the width ratio W1:W2 of the first region 10A and the second region 10B is between 1:1 and 20:1.

In addition, the textile 20x of the tissue scaffold 20 also has a plurality of pores P, and the size of the pores P may be between 100 micrometers and 800 micrometers. Furthermore, in accordance with some embodiments, the overall porosity of the textile 20x of the tissue scaffold 20 may be between 5% and 62%. In accordance with some embodiments, the porosity of the textile 20x of the tissue scaffold 20 in the first region 10A is greater than or equal to the porosity of the textile 20x in any second region 10B. In accordance with some embodiments, the porosity of the textile 20x of the tissue scaffold 20 in the first region 10A may be between 5% and 65%. In accordance with some embodiments, the porosity of the textile 20x of the tissue scaffold 20 in any second region 10B may be between 2% and 30%.

Similarly, the tissue scaffold 20 can have both mechanical strength and tissue compatibility, and is an ideal material for artificial ligaments, which can improve its ability to integrate with surrounding tissue and help fibroblasts and blood vessels grow around the tissue scaffold.

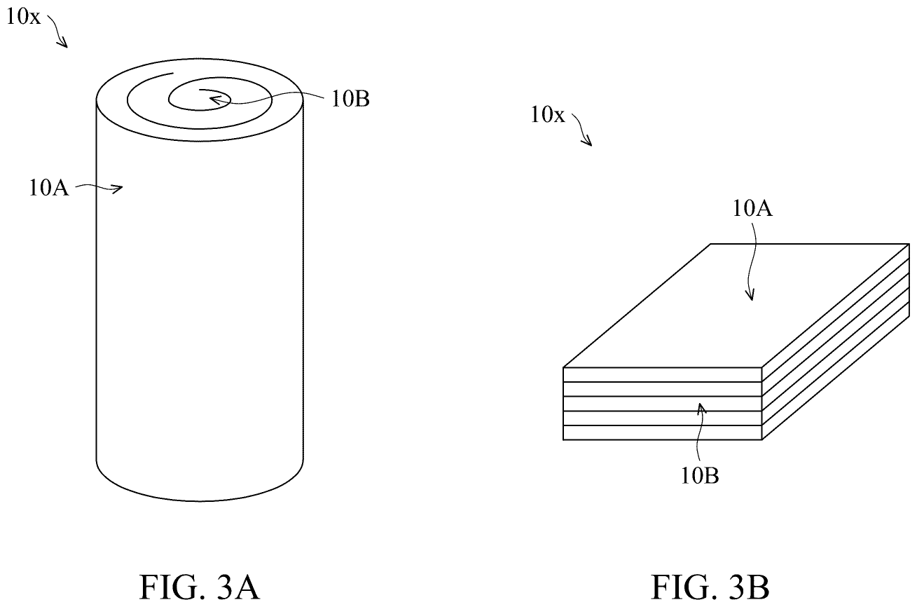

Next, please refer to FIG. 3A and FIG. 3B, which are schematic diagrams of tissue scaffolds in accordance with some other embodiments of the present disclosure. In accordance with some embodiments, the aforementioned textile 10x may be further rolled-up or folded into a multi-layer structure. As shown in FIG. 3A and FIG. 3B, in accordance with some embodiments, the multi-layer structure may have shapes such as columnar, strip-shaped, sheet-shaped, etc., but it is not limited thereto. For example, the columnar body may include a cylinder, a triangular prism, a quadrangular prism, a pentagonal prism or another suitably shaped column, but it is not limited thereto. Furthermore, in accordance with some embodiments, the first region 10A is located at the exterior of the multi-layer structure and the second region 10B is located at the interior of the multi-layer structure. In particular, the exposed first region 10A located at the exterior can provide a good environment for cell attachment and growth, while the second region 10B located on the interior has good mechanical strength and can provide structural stability, thus allowing the tissue scaffold to have both mechanical strength and tissue compatibility.

In addition, in accordance with some embodiments of the present disclosure, a method of manufacturing the aforementioned tissue scaffold is also provided. It should be understood that, in accordance with some embodiments, additional operations may be provided before, during and/or after the manufacturing method of the tissue scaffold described below, and may be understood in conjunction with the schematic diagram of the tissue scaffold in FIG. 1.

A method of manufacturing a tissue scaffold includes forming a textile 10x. Forming the textile 10x may include the following steps: providing a plurality of warp yarns 100 and performing warping, providing a plurality of weft yarns 200 and performing beat-up, and interweaving the warp yarns 100 and the weft yarns 200 to form the textile 10x.

In accordance with some embodiments, warping may be performed at a density of 10 to 100 warps/inch, for example, at 20 warps/inch, 30 warps/inch, 40 warps/inch, 50 warps/inch, 60 warps/inch, 70 warps/inch, 80 warps/inch or 90 warps/inch. In accordance with some embodiments, the warping length may extend indefinitely.

Furthermore, the warp yarns 100 have at least two different diameters. As mentioned above, the formed textile 10x includes a first region 10A and a second region 10B adjacent to the first region 10A, and the warp yarns 100 have different diameters in the first region 10A and the second region 10B. In accordance with some embodiments, the warp yarn 100-1 has a first diameter Da in the first region 10A, the warp yarn 100-2 has a second diameter Db in the second region 10B, and the first diameter Da is smaller than the second diameter Db. A plurality of warp yarns 100-2 having the second diameter Db may be arranged in sequence along the same first direction, and then a plurality of warp yarns 100-1 having the first diameter Da may be arranged in sequence along the first direction.

In accordance with some embodiments, the first diameter Da of the warp yarn 100-1 may be between 50 denier and 200 denier, but it is not limited thereto. In accordance with some embodiments, the second diameter Db of the warp yarn 100-2 may be between 500 denier and 2000 denier, but it is not limited thereto. Furthermore, in accordance with some embodiments, the filament number of warp yarns 100-1 in the first region 10A may be between 1F and 20F, but it is not limited thereto. In accordance with some embodiments, the filament number of warp yarns 100-2 in the second region 10B may be between 50F and 150F, but it is not limited thereto.

Moreover, in accordance with some embodiments, beat-up may be performed at a density of 10 weft picks/inch to 100 weft picks/inch, for example, at a density of 20 weft picks/inch, 30 weft picks/inch, 40 weft picks/inch, 50 weft picks/inch, 60 weft picks/inch 70 weft picks/inch, 80 weft picks/inch or 90 weft picks/inch. In accordance with some embodiments, beating up a first length is followed by empty beating for a second length. In accordance with some embodiments, the ratio of the first length to the second length is between 20:1 and 10:1, for example, 19:1, 18:1, 17:1, 16:1, 15:1, 14:1, 13:1, 12:1 or 11:1, but it is not limited thereto.

A plurality of weft yarns 200 may be arranged in sequence along a second direction, and the second direction is different from the first direction in which the warp yarns 100 are arranged. In accordance with some embodiments, the second direction in which the weft yarns 200 are arranged is substantially perpendicular to the first direction in which the warp yarns 100 are arranged.

As mentioned above, the weft yarns 200 have substantially the same diameter in the first region 10A and the second region 10B. In accordance with some embodiments, the diameter D2 of the weft yarn 200 may be between 50 denier and 200 denier, but it is not limited thereto. In accordance with some embodiments, the filament number of the weft yarn 200 may be between 1F and 20F, but it is not limited thereto.

In accordance with some other embodiments, a plurality of warp yarns 100-2 with the second diameter Db, a plurality of warp yarns 100-1 with the first diameter Da, and a plurality of warp yarns 100-2 with the second diameter Db may be arranged in sequence along the first direction, so that the tissue scaffold can further include another second region 10B, and the first region 10A is located between the two second regions 10B (as shown in FIG. 2).

In accordance with some other embodiments, after interweaving the warp yarns 100 and the weft yarns 200 to form the textile 10x, the textile 10x is further rolled-up or folded to have a multi-layer structure (as shown in FIG. 3A and FIG. 3B).

In addition, the formed textile 10x has a plurality of pores P, and the size of the pores P may be between 100 micrometers and 800 micrometers (μm). In accordance with some embodiments, the overall porosity of the formed textile 10x may be between 5% and 62%, but it is not limited thereto. In accordance with some embodiments, the porosity of the formed textile 10x in the first region 10A is greater than or equal to the porosity of the formed textile 10x in second region 10B. Furthermore, in accordance with some embodiments, in the formed textile 10x, the area of the first region 10A is larger than the area of the second region 10B, and the area ratio of the first region 10A to the second region 10B is between 1:1 and 20:1.

In order to make the above-mentioned and other purposes, features and advantages of the present disclosure more thorough and easy to understand, a number of examples and comparative examples are given below, and are described in detail as follows, but they are not intended to limit the scope of the present disclosure.

### Example 1: Preparation of Tissue Scaffold

500 D/96F high-strength polyethylene terephthalate (PET) yarn and 80 D/4F Y-shaped PET yarn were taken as warp yarns. Two strands of high-strength yarns were used as one warp, and one strand of Y-shaped yarn was used as one warp. The number of yarns was allocated according to the required diameter of the artificial ligament (according to the artificial ligament sample of the medical device manufacturer's Quality Management System (QMS), there were 40 warps of high-strength yarns on the left and right sides (corresponding to the second region), and 130 warps of Y-shaped yarns in the middle (corresponding to the first region). The warping was performed at a density of 50 warps/inch, and the warping length can be extended indefinitely. 80 D/4F Y-shaped PET/hydroxyapatite (Hap) fiber was taken as the weft yarn, and one strand was used as one weft. Beat-up was performed at a density of 50 weft picks/inch, leaving 3 cm of empty beating for every 40 cm of beat-up. Then, the warp yarns and weft yarns were interlaced to form a planar strip-shaped textile with a width of 10.6 cm.

### Example 2: Cell Growth Assessment of Tissue Scaffold

### 1. Cell Proliferation Analysis

First, the suspension of mesenchymal stem cells (mesenchymal stem cells isolated from human bone marrow) was planted on the textile surface of the tissue scaffold prepared in the aforementioned Example 1, and was left to stand for 1 hour in the incubator at 37° C. before adding culture medium (DMEM+10% serum) and culturing overnight. The cell culture medium was removed within 18 hours after the implanted cells have not replicated again. PrestoBlue (from Thermo Fisher Scientific PrestoBlue™ Cell Viability Reagent) was used to measure the number of cells attached to the culture plate. PrestoBlue was originally dark blue and non-fluorescent, and it was reduced to pink after the action of NADH dehydrogenase in mitochondria. By detecting fluorescence (Ex/Em: 560 nm/590 nm), the number of cells was reflected by establishing a standard curve based on the fluorescence value corresponding to the number of cells. Next, at the time points to be analyzed, the fluorescence changes were measured, the number of cells was quantitatively determined using the internal difference method based on the aforementioned standard curve, and the cell proliferation rate at each time point was calculated.

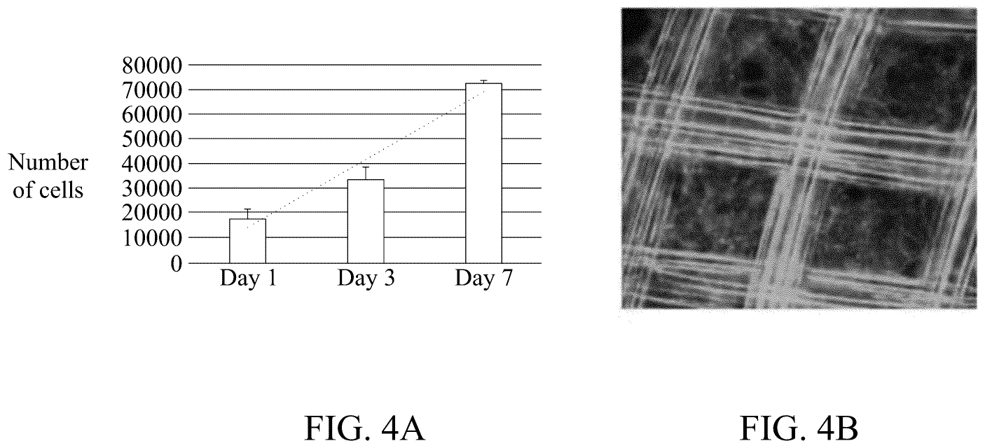

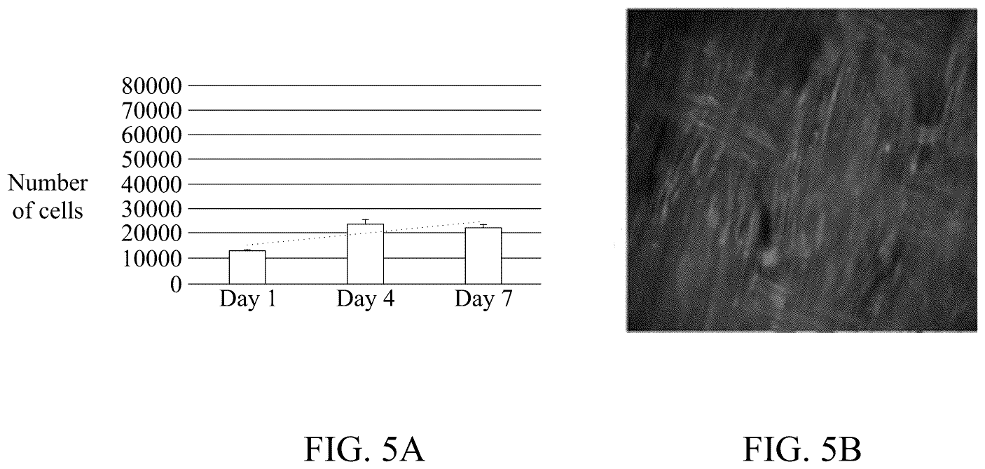

FIG. 4A and FIG. 5A respectively show the cell proliferation analysis results of the first region and the second region of the textile prepared in Example 1. As shown in FIG. 4A and FIG. 5A, compared with the second region using high-strength yarns as warp yarns, the number of cells growing in the first region using Y-shaped yarns as warp yarns was significantly greater, and the cell proliferation rate was higher. It can be seen from the results that the cell proliferation rate in the first region with smaller warp diameter and larger porosity was higher than the cell proliferation rate in the second region with larger warp diameter and smaller porosity. Specifically, on the 7th day of cell culture, the cell proliferation rate in the first region was about 3.5 to 4 times that of the second region.

### 2. Cell Morphology Analysis (α-Actin Staining)

The suspension of mesenchymal stem cells (mesenchymal stem cells isolated from human bone marrow) was planted on the textile surface, and was left to stand for 1 hour in the incubator at 37° C. before adding culture medium (DMEM+10% serum) and culturing overnight. The cell culture medium was removed within 18 hours after the implanted cells have not replicated again. Next, fixation was performed with 4% paraformaldehyde for more than 1 hour, then washed once with PBS, and 200 μl of 0.1% triton X100+1% BSA+5 μl of phalloidin (from Thermo Fisher Scientific) was added, and was washed once with PBS after 30 minutes of reaction. Then, 0.5 μM DAPI (from Roche. DAPI 4,6-Diamidine-2-phenylindole dihydrochloride) was added for staining for 5 minutes, and washed once with PBS and soaked in PBS. The cell morphology was observed with a fluorescence microscope (Zeiss Axiovert 200M). Phalloidin bound to fibrillar actin (a-actin). After fluorescent labeling, the distribution of intracellular microfilaments can be clearly seen under an optical microscope, thereby confirming the cell attachment morphology.

FIG. 4B and FIG. 5B respectively show the cell morphology (staining) analysis results of the first region and the second region of the textile prepared in Example 1. As shown in FIG. 4B and FIG. 5B, the cells in the first region using Y-shaped yarns as warp yarns can adhere and grow uniformly. In comparison, the cells in the second region using high-strength yarns as warp yarns are less conducive to cell growth.

### Example 3: Evaluation of the Relationship Between the Pores of Tissue Scaffold and Cell Growth

The textile of the tissue scaffold was prepared as described in Example 1, and the pore size of the textile was adjusted by interweaving at a density of 30 to 60 warps per inch, a density of 30 to 60 weft picks per inch, and using yarns of different thicknesses. Next, the effect of the pore size of the textile on cell growth was observed using the cell proliferation analysis and cell morphology analysis methods described in Example 2.

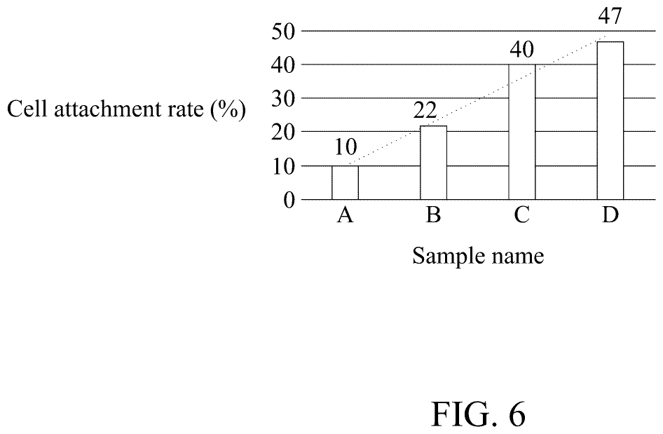

Table 1 and FIG. 6 show the quantification results of cell attachment rates of textile (in the first region) with different pore sizes. As shown in Table 1 and FIG. 6, the cell attachment rates of Sample B, Sample C and Sample D having pores were higher than that of Sample A without pores, and the pore size of the textile was substantially positively correlated with the cell attachment rate.

TABLE 1

| | Sample A | Sample B | Sample C | Sample D |
|---|---|---|---|---|
| side length of pore (μm) | 0 × 0 | 100 × 600 | 100 × 800 | 300 × 800 |
| area of pore (μm²) | 0 | 60000 | 80000 | 240000 |
| cell attachment rate (%) | 10 | 22 | 40 | 47 |

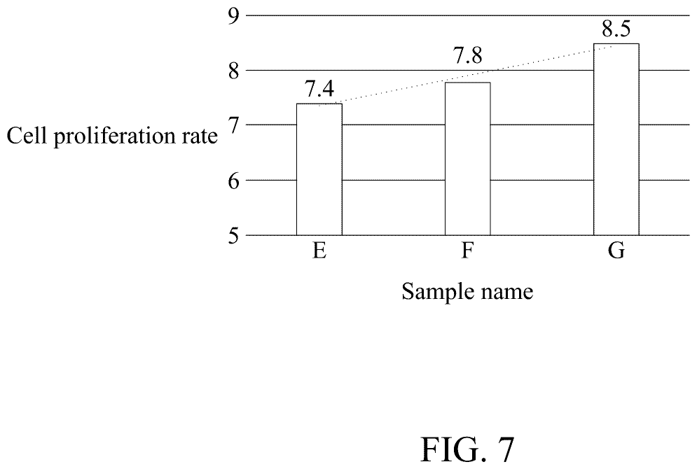

Table 2 and FIG. 7 show the results of cell proliferation rate of textiles with different pore sizes (in the first region). As shown in Table 2 and FIG. 7, the pore size of the textile was substantially positively correlated with the cell proliferation rate.

TABLE 2

| | Sample E | Sample F | Sample G |
|---|---|---|---|
| side length of pore (μm) | 100 × 300 | 200 × 300 | 600 × 300 |
| area of pore (μm²) | 30000 | 60000 | 180000 |
| cell proliferation rate (day 7/day 1) | 7.4 | 7.8 | 8.5 |

### Example 4: Evaluation of the Relationship Between the Multi-Layer Structure of Tissue Scaffold and Cell Growth

The textile of the tissue scaffold was prepared by the method as described in Example 1. The formed planar strip-shaped textile was further folded. The high-strength yarns on both sides were folded inward in half, then folded in half again so that the high-strength yarns (second region) were wrapped inside the Y-shaped PET yarns (first region). The textile was continuously folded in half to the required ligament diameter (according to the QMS system, a total of 3 folds, the diameter range was 2 mm-8 mm based on the required implantation site. The fold was fixed using needle sewing. The required length at the forth and back of the artificial ligament (16 cm according to the QMS system, and total length 35 cm) was left for cutting, and two 20 cm high-strength yarn binding ropes were sewed. Next, the effect of the multi-layered structure of the textile on cell growth was observed using the cell proliferation analysis and cell morphology analysis methods described in Example 2.

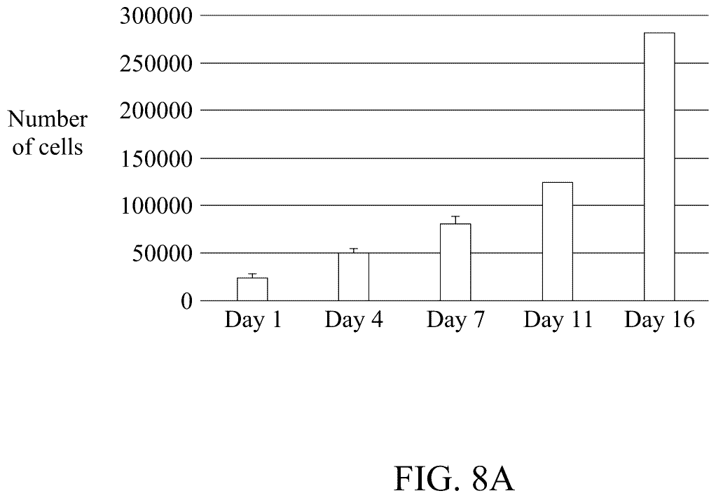

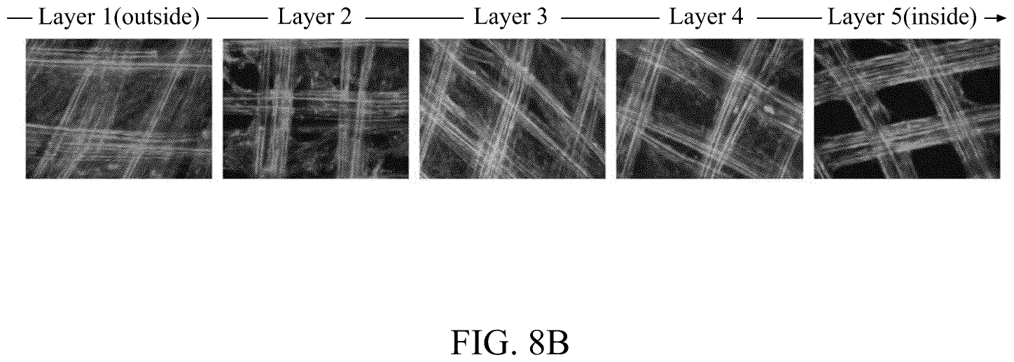

FIG. 8A and FIG. 8B respectively show the results of cell proliferation analysis and cell morphology analysis of the textile with a multi-layer structure (in the first region). As shown in FIG. 8A, from day 1 to day 16 of cell culture, the number of cells growing on the textile with the multi-layer structure continued to increase. On day 16 of cell culture, the cell growth rate was approximately achieved 9 to 10 times higher than that on day 1. Furthermore, as shown in FIG. 8B, there were cells growing from the outermost layer (marked as Layer 1) to the inner layer (marked as Layer 5) of the textile, and the number of cells growing gradually decreased from the outer layer to the inner layer. It can be seen that a textile with a multi-layered structure allows cells to grow across layers and increases its compatibility with tissue cells.

### Comparative Example 1: Evaluation of the Relationship Between the Arrangement of Tissue Scaffold and Cell Growth

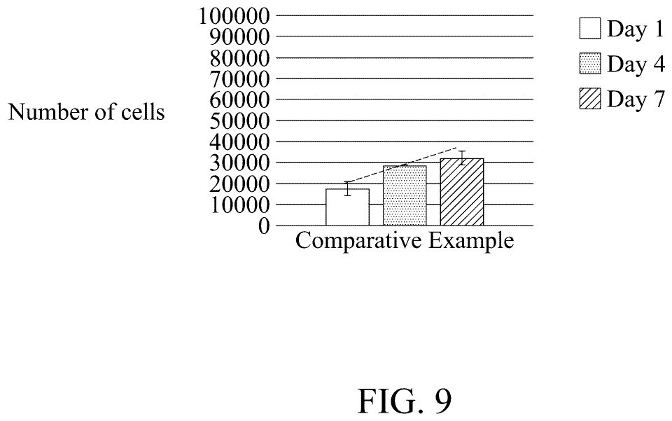

The textile of the tissue scaffold was prepared as described in Example 1, but the high-strength yarns and the Y-shaped yarns were alternately arranged (i.e., one high-strength yarn and one Y-shaped yarn were arranged alternately). Next, the cell growth of the textile was observed using the cell proliferation analysis method described in Example 2. FIG. 9 shows the results of cell proliferation analysis of the textile prepared in Comparative Example 1.

As shown in FIG. 9, the number of cell growth in the comparative example group only increased slightly, and the cell growth in the comparative example group was poor. It can be seen from the above results that when the textile does not have the first region and the second region, but has a structure in which high-strength yarns and Y-shaped yarns are alternately arranged, it is not conducive to cell growth.

### Example 5: Mechanical Strength Assessment of Tissue Scaffold

A tissue scaffold sample with a multi-layer structure was prepared as described in Example 4. The sample was then put into the clamp of the material testing machine (tensile and compression testing machine (brand: Instron, model: 4467)). The initial distance between the clamps was 20 cm. The sample was straightened and fixed in the clamp, and the central telescopic section of the sample should be located in the middle section of the clamp. The material testing machine performed tensile testing at a speed of 200 mm/min, and the displacement and tensile force data was recorded when the sample broke.

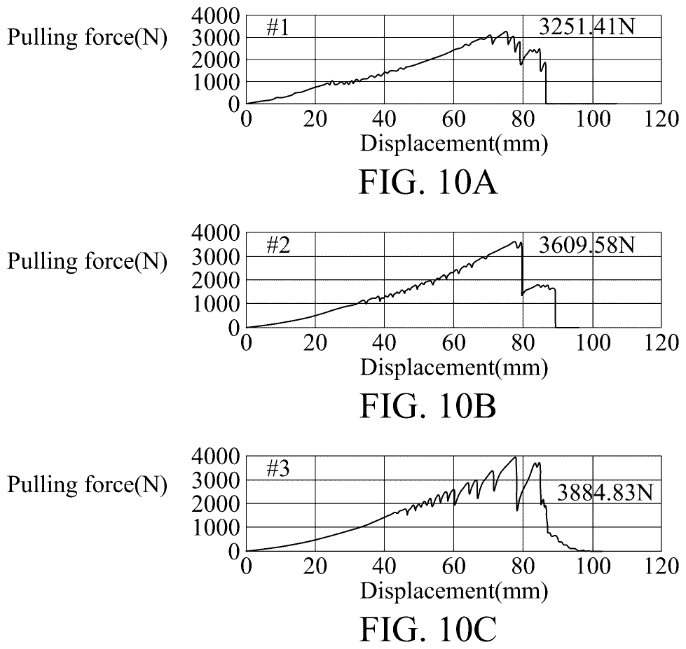

Table 3 and FIGS. 10A to 10C respectively show the tensile test results of the aforementioned tissue scaffold samples. Nos. 1 to 3 are the three test results of samples prepared in the same way. As shown in Table 3 and FIGS. 10A to 10C, the maximum tensile force of the tissue scaffold samples was stably greater than 3000 N, which was greater than the general load-bearing load of human joints (about 2000 N). It had good mechanical strength and was an ideal material for artificial ligaments.

TABLE 3

| No. | displacement (mm) | tensile force (N) |
|---|---|---|
| 1 | 75.26 | 3251.41 |
| 2 | 77.60 | 3609.58 |
| 3 | 77.76 | 3884.83 |

### Example 6: Application of Tissue Scaffold in Animal Testing

A tissue scaffold sample with a multi-layer structure was prepared as an artificial ligament by the method described in Example 4, and was used for anterior cruciate ligament reconstruction in pigs. First, hair was removed from the pig's hind limbs and then the knee joint was disinfected. After sterile measures and related equipment were prepared, a skin incision was made along the anterior of the knee joint and patella. The knee joint was opened and the anterior cruciate ligament was exposed. A 5 mm bone tunnel was drilled near the connection between the anterior cruciate ligament and the femoral condyle, and a 5 mm bone tunnel was drilled near the connection between the anterior cruciate ligament and the tibial condyle. The artificial ligament was passed through the bone tunnel and implanted at the anatomical position of the anterior cruciate ligament. The original anterior cruciate ligament was cut with a scalpel to simulate a complete rupture of the anterior cruciate ligament, and metal bone screw were locked into the bone tunnel to fix the ligament. Finally, the layers of tissue and skin were sutured.

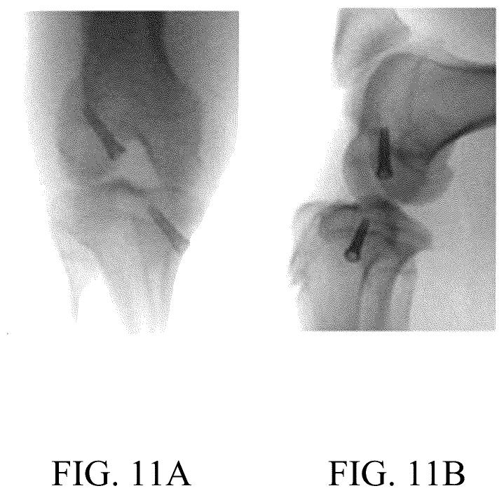

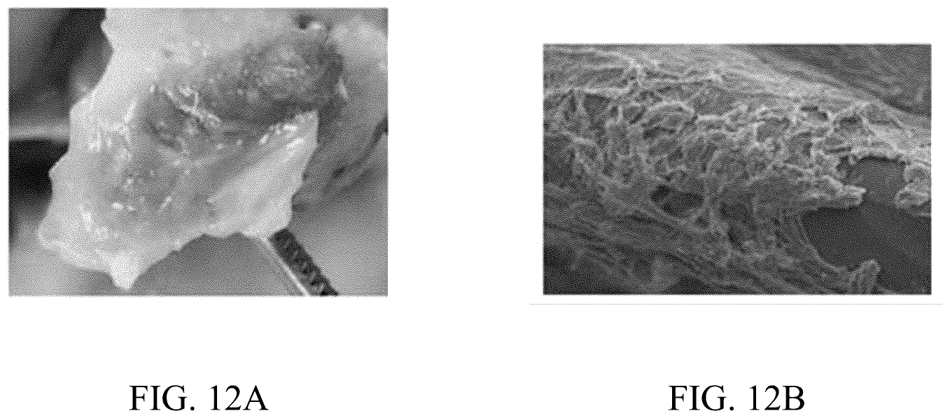

One month after the operation, the artificial ligament implantation position was observed with an X-ray detector (GE OEC FLUOROSTAR 7900). The results were as shown in FIG. 11A and FIG. 11B. The artificial ligament was well fixed on the anatomical position of the cruciate ligament, and the position was correct without abnormality. Furthermore, three months after the operation, the pigs were sacrificed and the artificial ligaments were removed. The cross-section and surface tissue growth of the artificial ligaments three months after they were implanted in the animals were observed with an electron microscope (ZEISS LEO 1530). As shown in FIG. 12A and FIG. 12B, many fibroblasts and blood vessels grew around the artificial ligament. The artificial ligament was well integrated with surrounding tissue cells, and many tissue cells covered the artificial ligament. It can be seen from the results that the tissue scaffold prepared in the Example has good tissue compatibility.

To summarize the above, the high-strength tissue scaffold provided in the embodiments of the present disclosure includes a textile with a specific configuration, so that the tissue scaffold has both mechanical strength and tissue compatibility, can effectively load a weight (for example, more than 3000 N) and provide structural stability, and has good tissue compatibility allowing regenerated cells to attach to tissue scaffold (for example, as artificial ligaments) and proliferate stably.

Although some embodiments of the present disclosure and their advantages have been described as above, it should be understood that various changes, substitutions and alterations can be made herein without departing from the spirit and scope of the disclosure as defined by the appended claims. In addition, each claim constitutes an individual embodiment, and the claimed scope of the present disclosure also includes the combinations of the claims and embodiments. The scope of protection of the present disclosure is subject to the definition of the scope of the appended claims.

What is claimed is:

1. A tissue scaffold, comprising a textile formed by interweaving a plurality of warp yarns and a plurality of weft yarns, the textile comprising:
a first region;
a second region adjacent to the first region, wherein the plurality of warp yarns have different diameters in the first region and the second region,
wherein the textile has a plurality of pores, and a size of each of the plurality of pores is between 100 μm and 800 μm.

2. The tissue scaffold as claimed in claim 1, wherein an area ratio of the first region to the second region is between 1:1 and 20:1.

3. The tissue scaffold as claimed in claim 1, wherein an overall porosity of the textile is between 5% and 62%.

4. The tissue scaffold as claimed in claim 1, wherein a porosity of the textile in the first region is greater than or equal to a porosity of the textile in the second region.

5. The tissue scaffold as claimed in claim 4, wherein the porosity of the textile in the first region is between 5% and 65%.

6. The tissue scaffold as claimed in claim 4, wherein the porosity of the textile in the second region is between 2% and 30%.

7. The tissue scaffold as claimed in claim 1, wherein the plurality of warp yarns have a first diameter in the first region and a second diameter in the second region, and the first diameter is smaller than the second diameter.

8. The tissue scaffold as claimed in claim 7, wherein the first diameter is between denier and 200 denier.

9. The tissue scaffold as claimed in claim 7, wherein the second diameter is between 500 denier and 2000 denier.

10. The tissue scaffold as claimed in claim 1, wherein a diameter of the plurality of weft yarns is between 50 denier and 200 denier.

11. The tissue scaffold as claimed in claim 1, wherein the materials of the plurality of warp yarns and the plurality of weft yarns comprise polyester, collagen, cellulose or a combination thereof.

12. The tissue scaffold as claimed in claim 1, wherein the tissue is rolled-up or folded into a multi-layered structure.

13. The tissue scaffold as claimed in claim 12, wherein the first region is located at exterior of the multi-layered structure and the second region is located at anterior of the multi-layered structure.

14. The tissue scaffold as claimed in claim 1, further comprising another second region, and the first region is located between the two second regions.

15. A method of manufacturing a tissue scaffold, comprising:
forming a textile, comprising:
providing a plurality of warp yarns and performing warping, wherein the plurality of warp yarns have at least two different diameters;
providing a plurality of weft yarns and performing beat-up; and
interweaving the plurality of warp yarns and the plurality of weft yarns to form the textile,
wherein the textile comprises a first region and a second region adjacent to the first region, wherein the plurality of warp yarns have different diameters in the first region and the second region, and the textile has a plurality of pores, and a size of the plurality of pores is between 100 μm and 800 μm.

16. The method of manufacturing a tissue scaffold as claimed in claim 15, wherein an area ratio of the first region to the second region is between 1:1 and 20:1.

17. The method of manufacturing a tissue scaffold as claimed in claim 15, wherein a porosity of the textile in the first region is greater than or equal to a porosity of the textile in the second region.

18. The method of manufacturing a tissue scaffold as claimed in claim 15, wherein the plurality of warp yarns have a first diameter in the first region and a second diameter in the second region, and the first diameter is smaller than the second diameter.

19. The method of manufacturing a tissue scaffold as claimed in claim 18, wherein the first diameter is between 50 denier and 200 denier.

20. The method of manufacturing a tissue scaffold as claimed in claim 18, wherein the second diameter is between 500 denier and 2000 denier.

21. The method of manufacturing a tissue scaffold as claimed in claim 15, wherein a diameter of the plurality of weft yarns is between 50 denier and 200 denier.

22. The method for manufacturing a tissue scaffold as claimed in claim 15, wherein warping is performed at a density of 10 to 100 warps/inch, and beat-up is performed at a density of 10 to 100 warp picks/inch.

23. The method of manufacturing a tissue scaffold as claimed in claim 15, further comprising rolling up or folding the textile to have a multi-layer structure.
